(19) United States
(12) Patent Application Publication
(10) Pub. No.: US 2016/0128356 A1
(43) Pub. Date: May 12, 2016

(54) PROCESS TO PRODUCE RICE BRAN HYDROLYSATES

(71) Applicant: DSM IP ASSETS B.V., Heerlen (NL)

(72) Inventors: Arthur Maurits Christiaan JANSE, Echt (NL); Sandjai SARDJOEPERSAD, Echt (NL); Gerardus Johannes Franciscus SMOLDERS, Echt (NL); Cecile VEERMAN, Echt (NL)

(21) Appl. No.: 14/890,471

(22) PCT Filed: May 8, 2014
(86) PCT No.: PCT/EP2014/059460

§ 371 Date: Nov. 11, 2015

(30) Foreign Application Priority Data
May 15, 2013 (EP) 13167819.5

(51) Int. Cl. A23J 3/34; C12P 21/00; A23J 3/14

(52) U.S. Cl. CPC A23J 3/346; A23J 3/14; C12P 21/00

(57) ABSTRACT

The present invention provides a (preferably defatted) rice bran hydrolysate composition which comprises of more than 50 wt % (on dry matter) of (poly)peptides and which has a DH (Degree of Hydrolysis) of at least 10%, preferably between 10 and 16% and more than 90%, preferably more than 95%, of the (poly) peptides has a molecular weight (MW) of more than 500 Da.

### FIELD OF THE INVENTION

The present invention relates to a process to extract proteins mildly from (preferably defatted) rice bran by limited proteolysis. The rice bran is washed before being subjected to limited proteolysis.

### BACKGROUND OF THE INVENTION

Protein extraction of agro-sources can be hampered by the solubility of the proteins itself or their interactions in the matrix with other constituents. The solubility is on its turn influenced by the processing steps before protein harvesting. For example, defatting of the material decreases the solubility of the proteins drastically. Therefore, the technique of proteolysis is applied to increase the solubility of the proteins and thus the protein extraction yield. The use of proteolytic enzymes mostly results in a bitter tasting product due to a high degree of hydrolysis with limited applications in food.

Rice bran is a by-product of the rice milling process. Generally rice milling yields about 15 weight percent (wt %) broken kernels, about 10 wt % rice bran, about 20 wt % hulls and about 55 wt % whole kernels. The typical protein content of rice bran is about 14 wt %. Other components in rice bran are moisture (about 10 wt %), crude oil (about 20 wt %), total dietary fiber (about 18 wt %), starch (about 22 wt %), ash (about 8 wt %) and other components (about 8 wt %). A valuable product obtained from the rice bran is rice bran oil which is the oil extracted from the germ and bran layer. After oil removal, a defatted product remains which is usually used in feed applications. The fat content of defatted rice bran is typically lower than 5 wt %. It is estimated that yearly more than 70 million ton of rice bran is produced, which still contains several valuable components, like proteins.

Extraction of rice bran proteins has been published in literature. Extraction methods include the use of water at alkaline conditions and/or the use of several carbohydrases (like amylases) sometimes in combination with the enzyme phytase. It is known that extraction of proteins from the non-treated rice bran is difficult. However, in case of defatted rice bran, protein extraction is even a more harsh task due to the heat treatment during oil extraction and especially during the toasting process to remove the residual hexane with protein denaturation as consequence.

US2011/0152180 describes bioactive pentapeptides from heat stabilized defatted rice bran. US 2011/0152180 uses Alcalase which is a very active enzyme and as a consequence leads to a strong protein degradation when incubated with different rice bran sources. According to example 1 of US 2011/0152180, the optimal degree of hydrolysis is 23.4%.

Silpradit et al (Optimization of rice bran protein hydrolysate production using Alcalase, 2010, As J Food Ag-Ind 3 (02), 221-231) describe conditions for rice bran hydrolysate production using Alcalase.

### SUMMARY OF THE INVENTION

The present invention provides a (preferably defatted) rice bran hydrolysate composition which comprises of more than 50 wt % (on dry matter) of (poly)peptides and which has a DH (Degree of Hydrolysis) of at least 10%, preferably between 10 and 16% and more than 90%, preferably more than 95%, of the (poly) peptides has a molecular weight (MW) of more than 500 Da.

The invention also provides a (preferably defatted) rice bran hydrolysate composition which comprises of more than 50 wt % (on dry matter) of (poly)peptides and which has a DH (Degree of Hydrolysis) of between 10 and 16% and more than 90%, preferably more than 95%, of the (poly) peptides has a molecular weight (MW) of more than 500 Da.

According to another aspect of the invention a process to produce a (preferably defatted) rice bran hydrolysate composition is provided which process comprises
adding an aqueous liquid, preferably water, to (preferably defatted) rice bran;
separating the liquid from the solid fraction to obtain a washed solid fraction;
adding an enzyme or enzyme composition to a suspension of the washed solid fraction which suspension has a concentration of between 5 and 30 wt %, preferably of between 12 and 30 wt %;
performing an enzyme incubation preferably at a pH between 6 and 8;
performing the enzyme incubation to an extent of hydrolysis of between a DH (Degree of Hydrolysis) of 10 and 16%;
performing the enzyme incubation at a temperature of between 30 and 80° C. preferably between 45 and 65° C., and
optionally separating the liquid from the solid fraction;
   whereby the enzyme or enzyme composition comprises an endoprotease.

The invention also provides a process to produce a (preferably defatted) rice bran hydrolysate composition having a protein content of more than 50 wt % (on dry matter) which process comprises
adding an aqueous liquid, preferably water, to (preferably defatted) rice bran;
separating the liquid from the solid fraction to obtain a washed solid fraction;
adding an enzyme or enzyme composition to a suspension of the washed solid fraction which suspension has a concentration of between 5 and 30 wt %, preferably of between 12 and 30 wt %;
performing an enzyme incubation preferably at a pH between 6 and 8;
performing the enzyme incubation to an extent of hydrolysis of between a DH (Degree of Hydrolysis) of 10 and 16%;
performing the enzyme incubation at a temperature of between 30 and 80° C. preferably between 45 and 65° C., and
optionally separating the liquid from the solid fraction;
   whereby the enzyme or enzyme composition comprises an endoprotease.

### DETAILED DESCRIPTION OF THE INVENTION

Throughout the present specification and the accompanying claims, the words “comprise” and “include” and variations such as “comprises”, “comprising”, “includes” and “including” are to be interpreted inclusively. That is, these words are intended to convey the possible inclusion of other elements or integers not specifically recited, where the context allows.

The articles “a” and “an” are used herein to refer to one or to more than one (i.e. to one or at least one) of the grammatical object of the article. By way of example, “an element” may mean one element or more than one element.

In the present invention, washing of source material (i.e. rice bran, preferably defatted rice bran) is combined with hydrolysis by proteases under such conditions that a rice bran protein hydrolysate is obtained which comprises of more than 50 wt % (on dry matter) (poly)peptides of which a low amount has a molecular weight (MW) of less than 500 Da.

The technique of washing and mild hydrolysis is preferably applied on defatted rice bran. However the process described is valid also/applicable for other rice bran sources such as raw rice bran and heat stabilized rice bran.

To valorize the (preferably defatted) rice bran or another agro material, a process is provided based on a limited or mild hydrolysis preferably followed by a solid/liquid separation. Both the liquid and the insoluble stream may be further dried. An advantage of this protease treatment followed by separation is that a solid fraction (pellet) is obtained with a high amount of total dietary fiber which can also be used in various food applications.

By rice bran is meant the hard outer layer of rice which consists of combined aleurone and pericarp. Along with germ, it is an integral part of whole rice, and is often produced as a by-product of milling in the production of refined rice. Raw rice bran is rice bran as obtained after milling. By defatted rice bran is meant rice bran of which at least part of the oil present is removed by for example extraction. Oil extraction is for example performed with hexane at approximately 60-65 degrees Celsius while the detoasting for hexane removal is typically performed at 110 degrees Celsius. By stabilized rice bran is meant rice bran which after milling is typically being stabilized at 130 degrees Celsius during<10 seconds.

Hydrolysis to extract proteins from various agro-sources is a well-known process. For rice bran, several studies exist to obtain the protein fraction using enzymes in addition to the more widely applied alkali extraction techniques see for example US20110305817. The use of proteases is generally more successful compared to the use of carbohydrases (see for example the review of Fabian and Ju (A Review on rice bran protein: its properties and extraction methods, Critical Reviews in Food Science and Nutrition, 2011, vol. 51, 816-827) summarizing enzymatic methods). Several proteases have been investigated in literature to obtain higher protein extraction yields (compared to carbohydrases). These hydrolysis products are usually bitter tasting hydrolysate compositions which cannot be widely applied in foods.

In the present invention washing of the (preferably defatted) rice bran and the use of a mild proteolysis technique on this washed rice bran results in a rice bran protein hydrolysate with increased wt % (on dry matter) of protein when compared to non-washed (preferably defatted) rice bran hydrolysates. The rice hydrolysate composition of the invention is widely applicable in various food applications such as beverages, bakery and dairy applications.

The present invention also relates to a process to produce a (preferably defatted) rice bran hydrolysate composition (preferably having a protein content of more than 50 wt % (on dry matter) which comprises
adding an aqueous liquid, preferably water, to (preferably defatted) rice bran;
separating the liquid from the solid fraction to obtain a washed solid fraction;
adding an enzyme or enzyme composition to a suspension of the washed solid fraction which suspension has a concentration of between 5 and 30 wt %, preferably of between 12 and 30 wt %;
performing an enzyme incubation preferably at a pH between 6 and 8;
performing the enzyme incubation to an extent of hydrolysis of between a DH(Degree of Hydrolysis) of 10 and 16%;
performing the enzyme incubation at a temperature of between 30 and 80° C. preferably between 45 and 65° C., and
optionally separating the liquid from the solid fraction;
   whereby the enzyme or enzyme composition comprises an endoprotease.

The present invention also relates to (preferably defatted) rice bran hydrolysate composition which contains of more than 90% of (poly) peptides with a molecular weight (MW) of more than 500 Da in combination with a degree of hydrolysis of at least 10%, preferably between 10 and 16%. Preferably the (preferably defatted) rice bran hydrolysate composition contains of more than 95% of (poly)peptides with a molecular weight of more than 500 Da.

The invention also provides a (preferably defatted) rice bran hydrolysate composition which comprises of more than 50 wt % (on dry matter) of (poly)peptides and which has a DH (Degree of Hydrolysis) of between 10 and 16% and more than 90%, preferably more than 95%, of the (poly) peptides has a molecular weight (MW) of more than 500 Da.

Apart from (poly)peptides the defatted rice bran hydrolysate composition may comprise carbohydrates, fat and minerals (determined often as ash fraction).

Preferably the (preferably defatted) rice bran hydrolysate composition is obtainable or is obtained by the process of the present invention. Preferably the content of di and tri-peptides present in the hydrolysate composition of the invention is less than 5 wt %. Preferably the content of free amino acids present in the hydrolysate composition of the invention is less than 2 wt %. Advantageously the rice bran used in the process of the invention is defatted rice bran and the hydrolysate composition obtained is a defatted rice bran hydrolysate composition.

Herein a “peptide” is defined as a chain of at least two amino acids that are linked through peptide bonds. A “polypeptide” is defined herein as a chain comprising of more than 30 amino acid residues and includes protein. As used herein a protein hydrolysate is a protein that has been hydrolysed by the action of a protease. A protease is an enzyme that hydrolyses peptide bonds between amino acids. A protein consists of one or more polypeptides, which consist of amino acids linked together by peptide bonds. A protein hydrolysate may be a protein that has been hydrolysed to a degree of hydrolysis (DH) of between 5 and 35%, for instance between 8 and 25% or between 10 and 16%, expressed as cleaved peptide bonds/total number of peptide bonds originally present×100%.

A (preferably defatted) rice bran hydrolysate composition is a composition which comprises at least 50 wt %, preferably at least 55 wt %, more preferably at least 60 wt % (on dry matter) of protein such as (poly)peptides and free amino acids and which is produced from (preferably defatted) rice bran.

According to the present invention an aqueous liquid, preferably water, is added to (preferably defatted) rice bran and subsequently the liquid is separated from the washed solid fraction. After the addition of water the separation can be done immediately or preferably the separation takes place after some time for example after at least 0.5 minute, at least 1 minute, at least 2 minutes, at least 5 minutes, at least 20 minutes, at least 60 minutes or between 0.5 minutes and 5 hours. During this time interval the water is advantageously mixed with the defatted rice bran. Hereafter the aqueous phase, solution or fraction may be separated from the solid phase or fraction in any convenient manner, such as by employing filtration and/or centrifugation. The liquid fraction will in general comprise compounds like carbohydrates and minerals (ash).

The invention therefore provides a process to produce a rice bran protein hydrolysate composition (preferably having a protein content of more than 50 wt % (on dry matter)) which comprises
adding an aqueous liquid, preferably water, to rice bran;
incubating said rice bran with said aqueous liquid for at least 0.5 minute, at least 1 minute, at least 2 minutes, at least 5 minutes, preferably at least 20 minutes, more preferably at least 60 minutes;
separating the liquid from the solid fraction to obtain a washed solid fraction;
adding an enzyme or enzyme composition to a suspension of the washed solid fraction which suspension has a concentration of between 5 and 30 wt %, more preferably of between 12 and 30 wt %;
performing an enzyme incubation preferably at a pH between 6 and 8;
performing the enzyme incubation to an extent of hydrolysis of between a DH (Degree of Hydrolysis) of 10 and 16%;
performing the enzyme incubation at a temperature of between 30 and 80° C. preferably between 45 and 65° C., and
optionally separating the liquid from the solid fraction;
   whereby the enzyme or enzyme composition comprises an endoprotease.

The invention also provides a process to produce a rice bran protein hydrolysate composition (preferably having a protein content of more than 50 wt % (on dry matter)) which comprises
adding an aqueous liquid, preferably water, to rice bran;
incubating, at a temperature of 4-80° C., preferably at room temperature, said rice bran with said aqueous liquid for at least 0.5 minute, at least 1 minute, at least 2 minutes, at least 5 minutes, preferably at least 20 minutes, more preferably at least 60 minutes;
separating the liquid from the solid fraction to obtain a washed solid fraction;
adding an enzyme or enzyme composition to a suspension of the washed solid fraction which suspension has a concentration of between 5 and 30 wt %, preferably of between 12 and 30 wt %;
performing an enzyme incubation preferably at a pH between 6 and 8;
performing the enzyme incubation to an extent of hydrolysis of between a DH (Degree of Hydrolysis) of 10 and 16%;
performing the enzyme incubation at a temperature of between 30 and 80° C. preferably between 45 and 65° C., and
optionally separating the liquid from the solid fraction;
   whereby the enzyme or enzyme composition comprises an endoprotease.

The invention also provides a process to produce a rice bran protein hydrolysate composition (preferably having a protein content of more than 50 wt % (on dry matter)) which comprises
adding an aqueous liquid, preferably water, to rice bran;
mixing, at a temperature of 4-80° C., preferably at room temperature, said rice bran with said aqueous liquid for at least 0.5 minute, at least 1 minute, at least 2 minutes, at least 5 minutes, preferably at least 20 minutes, more preferably at least 60 minutes;
separating the liquid from the solid fraction to obtain a washed solid fraction;
adding an enzyme or enzyme composition to a suspension of the washed solid fraction which suspension has a concentration of between 5 and 30 wt %, preferably of between 12 and 30 wt %;
performing an enzyme incubation preferably at a pH between 6 and 8;
performing the enzyme incubation to an extent of hydrolysis of between a DH (Degree of Hydrolysis) of 10 and 16%;
performing the enzyme incubation at a temperature of between 30 and 80° C. preferably between 45 and 65° C., and
optionally separating the liquid from the solid fraction;
   whereby the enzyme or enzyme composition comprises an endoprotease.

The invention further provides a process to produce a rice bran protein hydrolysate composition (preferably having a protein content of more than 50 wt % (on dry matter)) which comprises
adding an aqueous liquid, preferably water, to rice bran;
incubating, preferably mixing, at a temperature of 4-80° C., preferably at room temperature, and at a pH in the range of 4-8, said rice bran with said aqueous liquid for at least 0.5 minute, at least 1 minute, at least 2 minutes, at least 5 minutes, preferably at least 20 minutes, more preferably at least 60 minutes;
separating the liquid from the solid fraction to obtain a washed solid fraction;
adding an enzyme or enzyme composition to a suspension of the washed solid fraction which suspension has a concentration of between 5 and 30 wt %, preferably of between 12 and 30 wt %;
performing an enzyme incubation preferably at a pH between 6 and 8;
performing the enzyme incubation to an extent of hydrolysis of between a DH (Degree of Hydrolysis) of 10 and 16%;
performing the enzyme incubation at a temperature of between 30 and 80° C. preferably between 45 and 65° C., and
optionally separating the liquid from the solid fraction;
   whereby the enzyme or enzyme composition comprises an endoprotease.

More preferred pH ranges are 4-7, 5-7 or 6-7.

In a further preferred embodiment, said rice bran is defatted rice bran.

The used aqueous liquid is preferably water. Any kind of water, such as potable water, tap water, purified water (distilled water, double distilled water, deionized water or reverse osmosis water) can be used in a method of the invention. Moreover, the used water may comprise at least one added ingredient such as EDTA or citric acid. Such an ingredient is preferably added in low concentrations.

By the addition of water followed by the separation step increased protein content in the final rice bran hydrolysate composition can be obtained compared to a process without these steps. Advantageously the (preferably defatted) rice bran hydrolysate of the invention comprises of more than 50 wt % (on dry matter) of (poly)peptides. Without these steps the (preferably defatted) rice bran hydrolysate comprises in general of 35 to 42 wt % (on dry matter) of (poly)peptides.

An important aspect of our invention concerns that the protein fraction of the (defatted) rice bran is only partially hydrolysed. This is expressed in a relatively low degree of hydrolysis of for example between 10 and 16%. A higher degree of hydrolysis commonly results into bitter tasting products, while a low degree of hydrolysis results in low protein extraction yields. In our invention we apply a degree of hydrolysis which results in a product which can be easily formulated to have an acceptable taste and at the same time an acceptable protein extraction yield.

Enzymes, including proteases, are classified in the internationally recognized schemes for the classification and nomenclature of all enzymes from IUMB. The updated IUMB text for protease EC numbers can be found at the internet site: http://www.chem.qmul.ac.uk/iubmb/enzyme/index.html. In this system enzymes are defined by the fact that they catalyze a single reaction. This has the important implication that several different proteins are all described as the same enzyme, and a protein that catalyses more than one reaction is treated as more than one enzyme. The system categorises the proteases into endo- and exoproteases. Endoproteases are those enzymes that hydrolyze internal peptide bonds, exoproteases hydrolyze peptide bonds adjacent to a terminal a-amino group (“aminopeptidases”), or a peptide bond between the terminal carboxyl group and the penultimate amino acid (“carboxypeptidases”). The endoproteases are divided into sub-subclasses on the basis of catalytic mechanism. There are sub-subclasses of serine endoproteases (EC 3.4.21), cysteine endoproteases (EC 3.4.22), aspartic endoproteases (EC 3.4.23), metalloendoproteases (EC 3.4.24) and threonine endoproteases (EC 3.4.25).

An endoprotease used in the process of the invention to obtain a rice bran protein hydrolysate composition is advantageously a metallo endoprotease (EC. 3.4.24) such as Neutrase®, Maxazyme NNP DS® (EC. 3.4.24.28; bacillolysin), a serine endoprotease (EC 3.4.21) such as Alcalase® or Protease P®, or a cystein endopeptidase (EC 3.4.22) such as papain or bromelain. Preferably the endoprotease is a metallo endoprotease more preferably a bacillolysin (EC 3.4.24.28). Preferably, a process of the invention uses a metallo endoprotease, such as Maxazyme NNP DS.

Optionally, an aminopeptidase (EC 3.4.11) such as Corolase LAP® can be used in addition to an endoprotease to even further optimize the taste profile of the rice bran protein hydrolysate composition.

As mentioned before the (preferably defatted) rice bran concentration of the suspension used in the process of the invention is between 5 and 30 wt % or more preferably between 12 and 30 wt %. Preferably, the defatted rice bran concentration is between 15 and 25 wt %, more preferably the defatted rice bran concentration is between 15 and 22 wt %. In the literature, typical defatted rice bran concentrations are 10 wt % or less. However, those low values are hardly of industrial relevance due to the relatively high amounts of water that has to be removed in obtaining the final product. As mentioned before the incubation pH used in the enzyme incubation step of the invention is between 6 and 8. Preferably the incubation pH is between 6.5 and 7.5. Even more preferably, the incubation pH is between 7 and 7.5.

The incubation time used in the enzyme incubation step of the invention is in general between 1 and 6 hours. Preferably the incubation is between 1 and 4 hours. Even more preferably the incubation time is between 1 and 2 hours.

As mentioned before the incubation temperature of the enzyme incubation step is between 45 and 65° C. Preferably the incubation temperature is between 45 and 55° C. More preferably the incubation temperature is between 48 and 55° C.

According to another aspect of the invention the hydrolysate composition produced with the process of the invention is separated in a solid and liquid fraction using a solid/liquid separator. The aqueous phase, solution or fraction may be separated from the solid phase or fraction in any convenient manner, such as by employing filtration and/or centrifugation. The resulting liquid and/or the solid fraction may be dried in any convenient manner. Drying of the soluble fraction can be done for example in a spray drier, drum drier amongst other types. The solid or fiber fraction can be dried for example on drum drier, belt drier and other equipment which can handle high solids loadings.

After the hydrolysis of the invention the enzyme or enzyme composition can be inactivated. For example a heat shock can be applied.

Although the method of the invention can be performed on small scale, it is preferred to perform the claimed method on large scale with at least 2 liter suspension, more preferably at least 4 or 6 liter suspension and most preferred at least 8 or 10 liter suspension.

The product of the process of this invention comprises in general a protein content of between 50 wt % and 65 wt % on dry matter. The degree of hydrolysis of the (preferably defatted) rice bran hydrolysate composition is advantageously between 5 and 35%, for instance between 8 and 25% or between 10 and 20%. More preferably, the degree of hydrolysis is at least 10%, most preferably between 10 and 16%.

Functionalities like nitrogen solubility or foaming capacity and stability can be determined by a method as outlined in the experimental part herein.

The invention will now be elucidated with references to the following examples, without, however, being limited thereto.

### Methods and Materials

Materials

Defatted rice bran (DRB) is commercially available and can be obtained from raw rice bran by first hexane extraction at elevated temperatures 55-65° C. during less than 1 hour, followed by a so called toasting step at 105-110° C. to remove the residual hexane. The enzyme Maxazyme NNP DS® is a commercial product of DSM (The Netherlands) and is a metalloprotease.
EDTA: for analysis (99.7% pure), Merck KGaA Darmstadt, Germany.
Citrate: for analysis (>99% pure), Merck, KGaA, Darmstadt, Germany.

Protein Content

Protein content was determined by the Kjeldahl method according to AOAC Official Method 991.20 Nitrogen (Total) in Milk. A conversion factor of 6.25 was used to determine the amount of protein (% (w/w)).

Carbohydrate Content

Total carbohydrates were quantified by a modification of the NREL (National Renewable Energy Laboratory) method NREL/TP-510-42618. In contrast to this method, the detection, after hydrolysis, of the free mono saccharides was achieved by means of quantitative NMR (QNMR) with maleic acid as the internal standard. NMR spectra were recorded on a Bruker Avancelll 600 MHz NMR system equipped with a 5 mm cryo probe. The temperature of the probe was set to 280K.

SDS-PAGE

The peptide patterns were visualized by SDS-PAGE. All materials used for SDS-PAGE and staining were purchased from Invitrogen (Carlsbad, Calif., US). Samples were prepared using SDS buffer according to manufacturers instructions and separated on 12% Bis-Tris gels using MES-SDS buffer system according to manufacturers instructions. Staining was performed using Simply Blue Safe Stain (Collodial Coomassie G250

Nitrogen Solubility (NS %)

Protein solutions were prepared by dissolving protein powder at a protein concentration of 2% (w/w) in demineralized water. pH was adjusted to 4, 6.8 or 8.0 with 4M HCl or 4M NaOH (no additional salt was added).

Solutions were incubated for 2 hour at 50° C. while vigorous shaking. Subsequently, samples were centrifuged at 20.000 g for 5 min and supernatant was collected. The protein content of the supernatant and the protein powder samples was analyzed by the Kjeldahl method. Nitrogen solubility (NS %) was defined as:

$$NS \% = \frac{nitrogen in the supernatant ( mg )}{total nitrogen in a 100 mg sample} \times 100 \%$$

Foaming Capacity and Stability

Protein solutions were prepared by dissolving protein powder at a protein concentration of 2% (w/w) in demineralised water. The pH was adjusted to 4, 6.8 or 8 with 4M HCl or 4M NaOH. Foam was generated by vigorous whipping 100 g protein solution for 1 minute (Warning blender with 4 rotating blades at 18.000 rpm in a 1 L beaker). After foam generation, the foam/liquid content was transferred into 250 ml cylinders. Foaming capacity of proteins was determined by measuring the volume of the foam 30 seconds after preparing the foam. Foam stability was defined as the foam volume 30 minutes after preparation of the foam.

Dry Matter Content

Dry matter content was determined using infrared method at 105° C.

Degree of Hydrolysis

The degree of hydrolysis was determined with the rapid OPA-test (Nielsen, P. M., Petersen, D., Dambmann, C., Improved method for determining food protein degree of hydrolysis, Journal of Food Science 2001, 66, 642-646). The Kjeldahl factor used was 6.25.

Determination of Free Amino-acid Content

Samples were dissolved in a known amount of 0.1N HCL solution. 100 μl of this solution was mixed with an internal standard (IS) solution containing isotopic-labeled analogues of the amino acids to correct for ion-suppression effects from co-eluting peptides. 10 μl of this sample/IS solution was mixed with 70 μl Waters borate buffer and 20 μl Waters derivatization reagent. After mixing the solution was heated at 55° C. for 10 minutes. 1 μl was injected onto the UPLC-MS/MS system.

Analyses were performed on an Ultra high-Pressure Liquid Chromatograph combined with aXevo TQ mass spectrometer from Waters. The column was a Waters BEHC18 column (150×2.1 mm, 1.7μ) operating at a flow of 0.4 ml/min at 43° C. The mobile were from waters designated as AccQ-Tag Eluent A and AccQ-Tag Eluent B. Gradient was applied according to Table 1.

TABLE 1
Gradient profile

| Time (min) | Solvent A (%) | Solvent B (%) | Curve |
|---|---|---|---|
| 0 | 99.9 | 0.1 | |
| 1.14 | 99.9 | 0.1 | Linear |
| 2.00 | 98.5 | 1.5 | Linear |
| 5.50 | 98.1 | 1.9 | Linear |
| 6.50 | 98.0 | 2.0 | Convex |
| 10.00 | 97.6 | 2.4 | Linear |
| 12.00 | 96.0 | 4.0 | Linear |
| 20.00 | 88.0 | 12.0 | Linear |
| 35.00 | 85.0 | 15.0 | Convex |
| 36.00 | 2.0 | 98.0 | Linear |
| 38.00 | 2.0 | 98.0 | Linear |
| 39.00 | 99.9 | 0.1 | Linear |
| 60.00 | 99.9 | 0.1 | |

Amino acid derivates were ionized in positive mode using electrospray ionization. The amount of free amino acids was determined via an external calibration curve containing amino acids and isotopic labeled amino acids which were derivatized as described before.

Determination of Amount of Di and Tri-peptides

The amount di and tri-peptides was determined according to the following method:
Analysis with Mass Spectrometry (MS) of a mix of a known concentration of 10 different dipeptides and 10 different tripeptides
Analysis with MS of the provided sample: 1 ml solution of the sample (2 mg/ml) was centrifuged over a 3 kD spin column and the filtrate is analyzed.
Analysis of a blank sample (MQ)

The mixture of di and tri-peptides contained the following peptides:
Dipeptides:
GC, AG, FG, GP, RW, WL, VP, KK, AA, FL
Tripeptides:
WGP, GGP, YPP, LAL, LAV, EGP, LAK, LAW, VPL, NPI
(for the amino acids the 1-letter abbreviation is used)

The mass range of the smallest dipeptide (GG) to the largest tripeptide (WWW) is from 133-575 Da. Using this mass range the total peak area of the sample is determined. The peak area of the blank sample injection was subtracted from the sample injection to exclude the background ions.

Determination of the Molecular Weight Distribution

200 mg Rice bran hydrolysate composition sample was weighted in duplicate into 10 mL volumetric flasks and made up to volume with MQ-water. This suspension was mixed for 30 min at room temperature at 900 rpm with a magnetic stirrer.

Subsequently, 500 μL suspension was loaded on a 100 kDa cut-off filter (Pall nanosep 100 k omega) and centrifuged during 5 min at 20.000 g. Subsequently, the filtrate was loaded on a 30 kDa cut-off filter (Pall nanosep 30 K Omega) and centrifuged during 8 min at 20.000 g. Subsequently, the filtrate was loaded on a 3 kDa cut-off filter (Pall nanosep 3 K Omega) and centrifuged during 15 min at 20.000 g.

The residues on all three filters were re-dissolved in 500 μL MQ-water. A total of four samples were thus collected: 100 kDa residue (>100 kDa), 30 kDa residue (30-100 kDa), 3 kDa residue (3-30 kDa) and filtrate (˜450 μL) which passed all three filters (<3 kDa). Protein content was determined by Kjeldahl and the relative protein concentrations between all samples were calculated resulting in a molecular weight distribution.

Protein Extraction Yield

The protein extraction yield is defined as:

$$Yield \% = \frac{Total nitrogen in the supernatant}{Total nitrogen in dry start material} \times 100 \%$$

Alternatively, a protein extraction yield including water in pellet can be calculated by:

$$Yield ( including water in pellet ) \% = \frac{\begin{matrix}Total nitrogen in water phase including \\ the water in the pellet\end{matrix}}{total nitrogen in dry start material} \times 100 \%$$

### EXAMPLES

### Example 1

270 gram DRB (defatted rice bran) in 1530 gram potable water (15 wt %) of 15° C. was well-mixed during incubation at 15° C. for 1 hour. After 1 hour of incubation S/L separation was performed by centrifugation (Sorvall Evolution RC centrifuge with swing out rotor) was used, 5 min at 5000 RCF). 1187 gram of supernatant was removed. The 613 gram of solids was dissolved in 1140 gram potable water of 50° C. 0.75 ml of Maxazyme NNP DS® (4 ml/kg dry DRB) was added and incubated at pH 7.2, 50° C. for 2 hours. After 1 hour of incubation 0.38 ml of Maxazyme NNP DS (2 ml/kg dry DRB) was added once more. After in total 2 hours of incubation S/L separation was performed again. 1258 gram of supernatant was collected and analyzed for Protein content, Dry matter content and Degree of hydrolysis. Results:

| | Result |
|---|---|
| Dry matter | 2.18 wt % |
| N-Kjeldahl | 2490 [mg/kg] |
| Protein on dry matter (freeze dried sample) | 62 wt % |
| Degree of hydrolysis | 10.9% |

During the first S/L separation 8.2 wt % of the total Protein content was removed by the discharge of the supernatant.

The amount of (poly) peptides with a molecular weight (MW) of more than 500 Da was found to be more than 90 wt %.

As reference an experiment was performed without the well-mixed incubation step and DRB was directly hydrolyzed under hydrolysis conditions mentioned above. The reference hydrolysate composition had a protein dry matter content of 35-42%.

### Example 2

270 gram DRB (defatted rice bran) in 1530 gram potable water (15 wt %) of 50° C. was well-mixed during incubation at 50° C. for 1 hour. After 1 hour of incubation S/L separation was performed by centrifugation (Sorvall Evolution RC centrifuge with swing out rotor was used, 5 min at 5000RCF). 1133 gram of supernatant was removed. The 523 gram of the 572 gram of solids was dissolved in 1056 gram potable water of 50° C. 0.75 ml of Maxazyme NNP DS (4 ml/kg dry DRB) was added and incubated at pH 7.2, 50° C. for 2 hours. After 1 hour of incubation 0.38 ml of Maxazyme NNP DS (2ml/kg dry DRB) was added once more. After in total 2 hours of incubation S/L separation was performed again; 991 gram of supernatant was collected and analyzed for Protein content, Dry matter content and Degree of hydrolysis. Results:

| | Result |
|---|---|
| Dry matter | 2.53 wt % |
| N-Kjeldahl | 2490 [mg/kg] |
| Protein on dry matter | 61 wt % |
| Degree of hydrolysis | 11.5% |

During the first S/L separation 15.2% of the total Protein content was removed by the discharge of the supernatant.

The amount of (poly) peptides with a molecular weight (MW) of more than 500 Da was found to be more than 90 wt %.

### Example 3

270 gram DRB (defatted rice bran) in 1530 gram potable water (15 wt %) of 10° C. was well-mixed during incubation at 10° C. for 1 hour. After 1 hour of incubation S/L separation was performed by centrifugation (Sorvall Evolution RC centrifuge with swing out rotor was used, 5 min at 5000 RCF). 1142 gram of supernatant was removed. The 579 gram of the 616 gram of solids was dissolved in 772 gram potable water of 50° C. 0.79 ml of Maxazyme NNP DS (4 ml/kg dry DRB) was added and incubated at pH 7.2, 50° C. for 2 hours. After 1 hour of incubation 0.4 ml of Maxazyme NNP DS (2ml/kg dry DRB) was added once more. After in total 2 hours of incubation S/L separation was performed again. 797 gram of supernatant was collected and analyzed for Protein content, Dry matter content and Degree of hydrolysis. Results:

| | Result |
|---|---|
| Dry matter | 3.48 wt % |
| N-Kjeldahl | 3360 [mg/kg] |
| Protein on dry matter | 60.34 wt % |
| Degree of hydrolyses | 10.60% |

During the first S/L separation 12.6% of the total Protein content was removed by the discharge of the supernatant.

The amount of (poly) peptides with a molecular weight (MW) of more than 500 Da was found to be more than 90 wt %.

### Example 4

In a further example an analysis was being made to analyse the washed out material concerning the different constituents.

In this experiment, 202 gram DRB (defatted rice bran) was well mixed in 698 gram potable water (22wt %) of 15 C and incubated at this temperature of 15 C for 1 hour. After 1 hour of incubation S/L separation was performed by centrifugation (a Sorvall Evolution RC centrifuge with swing out rotor was used, 5 min at 5000 RCF). 513 Gram of supernatant was removed and 385 gram of solid was collected. With the supernatant 17% of the total sugars was removed and 7% of the N-Kjeldahl.

| | Mass | Dry matter | Sugars | N-Kjeldahl | Ash |
|---|---|---|---|---|---|
| | gram | (%) | [%] | [%] | % |
| DRB | 202 | 100% | 100% | 100% | 100% |
| | (water 698) | | | | |
| Sup | 513 | 10% | 17% | 7% | 7% |
| Pellet | 385 | 87% | 76% | 88% | 98% |
| Closure | 100% | 97% | 93% | 95% | 104% |

### Example 5

In this example, the washing of the defatted rice bran was shown with the following aqueous solutions 1 wt % Titripex III.2aq (EDTA, Merck) 1 wt % sodium citrate.2aq (Merck), pH not corrected (resulting in a pH of 6.5 of the suspension), pH 4 (corrected with HCl) and pH 2 (corrected with HCl).

Each addition was done in the following manner. 135 gram defatted rice bran was suspended in 765 gram water at ambient temperature during 1 hr under good mixing conditions. To this suspension the following additions were done in separate experiments: 9 gram EDTA, 9 gram citrate (tri-sodium citrate dihydraat), no addition, 25 g of 4N HCl, 45 g of 4 N HCl.

After centrifugation (as described in example 4), the following yields of dry matter and nitrogen to the supernatant phase were observed:

| | | | | pH | | |
|---|---|---|---|---|---|---|
| | | 1 wt % | 1 wt % | without | | |
| | Constituent | EDTA | citrate | correction | pH 4 | pH 2 |
| DRB | Dry matter | 100% | | | | |
| | Nitrogen | 100% | | | | |
| Supernatant | Dry matter | 21% | 20% | 12% | 28% | 29% |
| | Nitrogen | 19% | 12% | 8% | 10% | 10% |

1. A rice bran protein hydrolysate composition which comprises of more than 50 wt % (on dry matter) of (poly)peptides and which has a DH (Degree of Hydrolysis) of at least 10%, optionally between 10 and 16% and more than 90%, optionally more than 95%, of the (poly) peptides has a molecular weight (MW) of more than 500 Da.

2. A rice bran protein hydrolysate composition of claim 1 which has a content of free amino acids of less than 2 wt % on dry matter.

3. A rice bran protein hydrolysate composition of claim 1 which is a defatted rice bran protein hydrolysate.

4. A process to produce a rice bran protein hydrolysate composition which comprises
adding an aqueous liquid, optionally water, to rice bran;
separating the liquid from the solid fraction to obtain a washed solid fraction;
adding an enzyme or enzyme composition to a suspension of the washed solid fraction which suspension has a concentration of between 5 and 30 wt %, optionally of between 12 and 30 wt %;
performing an enzyme incubation optionally at a pH between 6 and 8;
performing the enzyme incubation to an extent of hydrolysis of between a DH (Degree of Hydrolysis) of 10 and 16%;
performing the enzyme incubation at a temperature of between 30 and 80° C. optionally between 45 and 65° C.; and
optionally separating the liquid from the solid fraction;
whereby the enzyme or enzyme composition comprises an endoprotease.

5. A process to produce the rice bran protein hydrolysate composition according to claim 6 wherein the endoprotease is a metallo endoprotease optionally a bacillolysin (EC 3.4.24.28).

6. A process to produce the hydrolysate composition according to claim 4 wherein said rice bran is defatted rice bran.

7. A process according to claim 4, wherein said rice bran is incubated with said aqueous liquid at a pH 4-8.

8. A process according to claim 4, wherein said rice bran is incubated with said aqueous liquid at a temperature of 4-80° C., optionally at room temperature.

9. A process according to claim 4, wherein said rice bran is incubated with said aqueous liquid for at least 0.5 minute, at least 1 minute, at least 2 minutes, optionally at least 5 minutes, optionally at least 20 minutes, optionally at least 60 minutes.

10. A process according to claim 4, wherein said rice bran is mixed with said aqueous liquid.

11. A process according to claim 4, wherein said rice bran protein hydrolysate is a rice bran protein hydrolysate having a protein content of more than 50 wt % (on dry matter).

12. A rice bran protein hydrolysate obtainable by the process of claim 4.
